(19) United States
(12) Patent Application Publication
(10) Pub. No.: US 2025/0325739 A1
(43) Pub. Date: Oct. 23, 2025

(54) CONTROL OF CARBON DIOXIDE TRANSFER IN OXYGENATOR FOR EXTRACORPOREAL BLOOD GAS EXCHANGE

(71) Applicant: Maquet Critical Care AB, Solna (SE)

(72) Inventors: Åke LARSSON, Järfälla (SE); Mario LONCAR, Ekerö (SE)

(21) Appl. No.: 18/872,160

(22) PCT Filed: Jun. 5, 2023
(86) PCT No.: PCT/SE2023/050560

§ 371 Date: Dec. 5, 2024

(30) Foreign Application Priority Data
Jun. 9, 2022 (SE) 2250693-5

(51) Int. Cl. A61M 1/36; A61B 5/1455; A61M 1/16; A61M 16/00

(52) U.S. Cl. CPC A61M 1/3666; A61B 5/14557; A61M 1/1698; A61M 16/022; A61M 2205/05; A61M 2205/3334; A61M 2205/3368; A61M 2230/202; A61M 2230/205

(57) ABSTRACT

A method for controlling carbon dioxide [CO2] removal in a device for extracorporeal blood gas exchange is disclosed. The device comprises an oxygenator including a membrane acting as a gas-liquid barrier enabling CO2 transfer between a bloodstream and a sweep gas flow through the oxygenator. The method comprises the steps of measuring a pre-oxygenator fraction of CO2 [FCO2ᵢₙ] in the sweep gas flow upstream of the oxygenator, measuring a pre-oxygenator sweep gas flow rate (\dot{V}ᵢₙ) of the sweep gas flow upstream of the oxygenator, measuring a post-oxygenator fraction of CO2 [FCO2ₒᵤₜ] in the sweep gas flow downstream of the oxygenator, measuring a post-oxygenator sweep gas flow rate (\dot{V}ₒᵤₜ) of the sweep gas flow downstream of the oxygenator, and calculating a net CO2 exchange [\dot{V}CO2ₙₑₜ] over the membrane based on measured FCO2ᵢₙ, \dot{V}ᵢₙ, FCO2ₒᵤₜ and \dot{V}ₒᵤₜ.

### TECHNICAL FIELD

The present disclosure relates to the field of extracorporeal blood gas exchange by use of an oxygenator for extracorporeal removal of carbon dioxide from the blood of a patient. In particular, the disclosure relates to a method for controlling carbon dioxide exchange over the oxygenator through addition of carbon dioxide to a sweep gas flow through the oxygenator.

### BACKGROUND

Mechanical ventilators and medical devices for oxygenation and extracorporeal removal of CO2 from human blood are well known examples of intensive care equipment that are used to provide ventilatory and sometimes circulatory support to patients with reduced lung function.

Mechanical ventilators are used to provide respiratory treatment to patients through the supply of oxygen-containing breathing gas to the patient's lungs, allowing CO2 to be removed from, and oxygen to be added to, the circulatory system of a patient through gas exchange within the lungs.

Historically, medical devices for extracorporeal removal of CO2 from human blood, often referred to as extracorporeal membrane oxygenation (ECMO) devices, have primarily been used to provide ventilatory and circulatory support to patients having reduced lung and/or heart function in situations where conventional and less invasive treatments, such as mechanical ventilation, have been insufficient. Lately, however, combined treatment by ECMO devices and mechanical ventilators have gained more and more interest from clinicians also in the treatment of patients suffering from less severe lung conditions.

In an ECMO device, carbon dioxide rich blood is withdrawn from the patient and provided to an oxygenator that serves as an artificial lung by removing CO2 and adding oxygen to the blood before the oxygen-enriched blood is returned to the circulatory system of the patient. The removal of CO2 and the addition of oxygen is achieved by sweeping an oxygen-containing sweep gas flow through the oxygenator, allowing gas exchange between the blood and the sweep gas to take place over the oxygenator membrane.

The sweep gas flow is typically a flow of oxygen and/or air. The degree of CO2 removal by the oxygenator may, in this case, be controlled by regulating the sweep gas flow and/or the fraction of oxygen in the sweep gas flow, as described in e.g. US20150034082.

It is also known to add carbon dioxide to the sweep gas flow to adjust the degree of CO2 removal by the oxygenator without affecting the addition of oxygen to the blood of the patient. In particular, it allows an ongoing ventilatory treatment provided by the mechanical ventilator and/or a lung function of the patient to be evaluated by minimizing CO2 removal by the oxygenator with reduced risk of blood hyperventilation and improved pH control. Such an evaluation is often referred to as a weaning test since it is sometimes performed to assess the patient's readiness to be weaned from the ECMO device and/or the mechanical ventilator.

Normally, in order to minimize CO2 removal by the oxygenator through addition of CO2 to the sweep gas flow, the clinician manually adjusts the addition of CO2 until a measured fraction of CO2 in the sweep gas flow upstream of the oxygenator equals a measured fraction of CO2 in the sweep gas flow downstream of the oxygenator. When the fraction of CO2 upstream the oxygenator matches the fraction of CO2 downstream the oxygenator, it is assumed that no CO2 removal takes place over the oxygenator.

However, this assumption is often erroneous due to, e.g., the so called Haldane effect, according to which oxygenation of blood causes displacement of CO2 from haemoglobin, thereby increasing the removal of CO2.

Furthermore, the manual control of the addition of CO2 to the sweep gas flow increases the manual workload of the clinician and the risk of human errors in the treatment of the patient.

### SUMMARY

It is an object of the present disclosure to present a method, a computer program and a system for solving or mitigating one or more of the above mentioned problems associated with the prior art.

It is a particular object of the present disclosure to present a method, a computer program and a system for improved control of addition of CO2 to an oxygenator sweep gas flow, both in terms of accuracy and automation.

This and other objects, which will become apparent in view of the detailed description following hereinafter, are achieved in accordance with a first aspect of the present disclosure by a method, a computer program and a system as set forth below.

According to this first aspect of the disclosure, there is provided a method for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator. The method comprises the steps of:
adding CO2 to the sweep gas flow upstream of the oxygenator to control a degree of CO2 removal from the bloodstream by the oxygenator,
determining a measure of CO2 removal by the oxygenator based on a difference (ΔCCO2_{blood}) between a measure of a pre-oxygenator content of CO2 (CCO2ᵢₙ) in the bloodstream upstream of the oxygenator and an estimate of a post-oxygenator content of CO2 (CCO2ₒᵤₜ) in the bloodstream downstream of the oxygenator, and
utilizing the measure of CO2 removal for improved regulation of the CO2 addition to the sweep gas flow.

By determining the measure of CO2 removal as a difference ΔCCO2_{blood} between a measure of CCO2ᵢₙ in the bloodstream upstream of the oxygenator and an estimate of CCO2ₒᵤₜ in the bloodstream downstream of the oxygenator, a more correct measure of CO2 removal can be obtained compared to a solution where CO2 addition is controlled, e.g., based on a difference between measured FCO2 upstream and downstream of the oxygenator. Thus, the proposed technique offers an accurate yet relatively non-complex approach for controlling a degree of CO2 removal by the oxygenator.

According to some embodiments, the step of utilizing the measure of CO2 removal for improved regulation of the addition of CO2 to the sweep gas flow comprises:
presenting the measure of CO2 removal to an operator of the device as decision support in manual adjustment of the addition of CO2 to the sweep gas flow, and/or
presenting, to the operator, a recommendation for adjustment of the addition of CO2 to the sweep gas flow, based on the measure of CO2 removal and a set target for CO2 removal by the oxygenator, and/or
automatically regulating the addition of CO2 to the sweep gas flow based on the measure of CO2 removal and a set target for CO2 removal by the oxygenator.

By presenting the measure of CO2 removal to an operator of the device, improved manual regulation of the addition of CO2 can be obtained, allowing the operator to adjust the addition of CO2 to the sweep gas flow to obtain a desired degree of CO2 removal, e.g., zero CO2 removal.

Recommendations for adjustment of the addition of CO2 may, for example, be based on the sign and/or magnitude of ΔCCO2_{blood}. In one example, the recommendation may be a recommendation for adjustment of a measured fraction of CO2 (FCO2ᵢₙ) in the sweep gas flow upstream of the oxygenator by a certain amount or to a recommended level.

Automatic regulation of the addition of CO2 may be performed based on the measure of CO2 removal and a set target value for CO2 removal. In particular, automatic regulation of CO2 removal may be advantageous in situations where no CO2 removal by the oxygenator is desired. In such a scenario, the CO2 addition may be regulated using a closed control loop striving to reach and/or maintain a ΔCCO2_{blood} of zero.

According to some embodiments, the measure of CO2 removal is determined from pre-oxygenator measurements of partial pressures of CO2 (PCO2ᵢₙ) and O2 (PO2ᵢₙ) in the bloodstream upstream of the oxygenator, and post-oxygenator measurements of fractions of CO2 (FCO2ₒᵤₜ) and O2 (FO2ₒᵤₜ) in the gas sweep flow downstream of the oxygenator. Measurements of pre-oxygenator PCO2 and PO2 may be obtained by a standard blood gas analyser (BGA). A BGA is normally used during ECMO treatment and hence readily available at the bedside of the patient. By taking both pre-oxygenator and post-oxygenator CO2 and O2 into account in the determination of the measure of CO2 removal, the Haldane effect may be compensated for and a more reliable measure of CO2 removal can be obtained.

The first aspect of the present disclosure hence represents a semi gas-based approach for determining a measure of CO2 removal by the oxygenator, where bloodgas analysis of the bloodstream upstream of the oxygenator is combined with gas analysis of the sweep gas flow downstream of the oxygenator.

According to some embodiments, the method comprises the steps of:
measuring a pre-oxygenator partial pressure of CO2 (PCO2ᵢₙ) in the bloodstream upstream of the oxygenator,
measuring a pre-oxygenator partial pressure of O2 (PO2ᵢₙ) in the bloodstream upstream of the oxygenator,
measuring a post-oxygenator fraction of CO2 (FCO2ₒᵤₜ) in the sweep gas flow downstream of the oxygenator,
measuring a post-oxygenator fraction of O2 (FO2ₒᵤₜ) in the sweep gas flow downstream of the oxygenator,
estimating a post-oxygenator partial pressure of CO2 (PCO2ₒᵤₜ) and a post-oxygenator partial pressure of O2 (PO2ₒᵤₜ) in the bloodstream downstream of the oxygenator based on FCO2ₒᵤₜ and FO2ₒᵤₜ, and determining the difference ΔCCO2_{blood} between CCO2ᵢₙ and CCO2ₒᵤₜ based on PCO2ᵢₙ, PO2ᵢₙ, PCO2ₒᵤₜ and PO2ₒᵤₜ.

The measure of the pre-oxygenator content of CO2 (CCO2ᵢₙ) in the bloodstream upstream of the oxygenator may be expressed as a function of PCO2ᵢₙ, PO2ᵢₙ, T_{in,blood} and Hbᵢₙ, where T_{in,blood} is the temperature of the bloodstream upstream of the oxygenator and Hbᵢₙ is the haemoglobin content of the bloodstream upstream of the oxygenator. Likewise, the estimate of the post-oxygenator content of CO2 (CCO2ₒᵤₜ) may be expressed as a function of PCO2ₒᵤₜ PO2ₒᵤₜ, T_{out,blood} and Hbₒᵤₜ, where T_{out,blood} is the temperature of the bloodstream downstream of the oxygenator and Hbₒᵤₜ is the haemoglobin content of the bloodstream downstream of the oxygenator. Since Hbᵢₙ can be assumed to correspond to Hbₒᵤₜ, and since T_{in,blood} can be assumed to be relatively close to T_{out,blood}, those quantities need not be taken into account in an approximate determination of the difference ΔCCO2_{blood} between CCO2ᵢₙ and CCO2ₒᵤₜ.

According to some embodiments, the method comprises the steps of:
measuring or estimating a pre-oxygenator temperature (T_{in,blood}) of blood in the bloodstream upstream of the oxygenator,
measuring or estimating a post-oxygenator temperature (T_{out,blood}) of blood in the bloodstream downstream of the oxygenator, and
determining the difference ΔCCO2_{blood} between CCO2ᵢₙ and CCO2ₒᵤₜ based on PCO2ᵢₙ, PO2ᵢₙ, T_{in,blood}, PCO2ₒᵤₜ, PO2ₒᵤₜ and T_{out,blood}

By taking the pre-oxygenator and the post-oxygenator temperatures of the bloodstream into account in the determination of ΔCCO2_{blood}, a more accurate measure of CO2 removal can be determined.

According to some embodiments, the method comprises the steps of:
measuring or estimating a haemoglobin content (Hb) of blood in the bloodstream through of the oxygenator, and
determining the difference ΔCCO2_{blood} between CCO2ᵢₙ and CCO2ₒᵤₜ based on PCO2ᵢₙ, PO2ᵢₙ, T_{in,blood}, PCO2ₒᵤₜ, PO2ₒᵤₜ, T_{out,blood}, and Hb.

By measuring and taking Hb into account in the determination of ΔCCO2_{blood}, the transfer of CO2 over the oxygenator membrane can be quantified and an actual net CO2 exchange, VCO2ₙₑₜ, in ml/min can be calculated and used as the measure of CO2 removal. This is advantageous in that a metric indicating actual CO2 removal in ml/min over the membrane can be presented to an operator of the device, or be used as a control parameter in manual, semi-automatic or automatic control of the sweep gas flow rate and/or an addition of CO2 to the sweep gas flow, in order to meet a set target value for net CO2 exchange.

Accordingly, the method may comprise the steps of:
calculating a net CO2 exchange (\dot{V}CO2ₙₑₜ) over the membrane based on ΔCCO2_{blood}, and
utilizing \dot{V}CO2ₙₑₜ as the measure of CO2 removal.

According to some embodiments, the method comprises the steps of:
receiving a target value for the measure of CO2 removal, and
automatically regulating the addition of CO2 to the sweep gas flow so as to reach and/or maintain the target value for the measure of CO2 removal.

According to some embodiments, the device is connected to a patient who is also connected to a mechanical ventilator for mechanically ventilating the patient through the supply of breathing gas to the lungs of the patient, wherein the target value is set to zero in order to evaluate a ventilatory treatment provided by the mechanical ventilator and/or a lung function of the patient.

The above mentioned features allow a so called weaning test of the patient to be accurately and automatically performed. By ensuring that the oxygenator does not participate in the removal of CO2 from the patient's blood, the ventilatory treatment and/or the lung function of the patient can be reliably evaluated.

According to the first aspect of the disclosure, there is also provided a computer program for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator. The computer program comprises computer-readable instructions which, when executed by a control computer, causes the above described method to be performed.

According to the first aspect of the disclosure, there is also provided a computer program product comprising a non-transitory memory hardware device storing a computer program for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator The computer program comprises computer-readable instructions which, when executed by a control computer, causes the above described method to be performed.

According to the first aspect of the disclosure, there is also provided a system for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator. The system further comprises a sweep gas regulator for adding CO2 to the sweep gas flow upstream of the oxygenator in order to control a degree of CO2 removal from the bloodstream by the oxygenator. The device further comprises at least one control computer configured to:
determine a measure of CO2 removal by the oxygenator based on a difference (ΔCCO2_{blood}) between a measure of a pre-oxygenator content of CO2 (CCO2ᵢₙ) in the bloodstream upstream of the oxygenator and an estimate of a post-oxygenator content of CO2 (CCO2ₒᵤₜ) in the bloodstream downstream of the oxygenator, and
utilizing the measure of CO2 removal for improved regulation of the CO2 addition to the sweep gas flow.

According to some embodiments, the at least one control computer is configured to utilize the measure of CO2 removal for improved regulation of the CO2 addition to the sweep gas flow by:
causing the measure of CO2 removal to be presented to an operator of the device as decision support in manual adjustment of the addition of CO2 to the sweep gas flow, and/or
causing a recommendation for adjustment of the addition of CO2 to the sweep gas flow to be presented to an operator of the device, which recommendation is based on the measure of CO2 removal and a set target for CO2 removal by the oxygenator, and/or
automatically regulating the addition of CO2 to the sweep gas flow based on the measure of CO2 removal and a set target for CO2 removal by the oxygenator.

According to some embodiments, the control computer is configured to determine the measure of CO2 removal from pre-oxygenator measurements of partial pressures of CO2 (PCO2ᵢₙ) and O2 (PO2ᵢₙ) in the bloodstream upstream of the oxygenator, and post-oxygenator measurements of fractions of CO2 (FCO2ₒᵤₜ) and O2 (FO2ₒᵤₜ) in the gas sweep flow downstream of the oxygenator.

According to some embodiments, the control computer is configured to:
receive a measurement of a pre-oxygenator partial pressure of CO2 (PCO2ᵢₙ) in the bloodstream upstream of the oxygenator,
receive a measurement of a pre-oxygenator partial pressure of O2 (PO2ᵢₙ) in the bloodstream upstream of the oxygenator,
receive a measurement of a post-oxygenator fraction of CO2 (FCO2ₒᵤₜ) in the sweep gas flow downstream of the oxygenator,
receive a measurement of a post-oxygenator fraction of O2 (FO2ₒᵤₜ) in the sweep gas flow downstream of the oxygenator,
estimate a post-oxygenator partial pressure of CO2 (PCO2ₒᵤₜ) and a post-oxygenator partial pressure of O2 (PO2ₒᵤₜ) in the bloodstream downstream of the oxygenator based on FCO2ₒᵤₜ and FO2ₒᵤₜ, and
determine the difference ΔCCO2_{blood} between CCO2ᵢₙ and CCO2ₒᵤₜ based on PCO2ᵢₙ, PO2ᵢₙ, PCO2ₒᵤₜ, and PO2ₒᵤₜ.

According to some embodiments, the control computer is configured to:
estimate or receive a measurement of a pre-oxygenator temperature (T_{in,blood}) of blood in the bloodstream upstream of the oxygenator,
estimate or receive a measurement of a post-oxygenator temperature (T_{out,blood}) of blood in the bloodstream downstream of the oxygenator, and
determine the difference ΔCCO2_{blood} between CCO2ᵢₙ and CCO2ₒᵤₜ based on PCO2ᵢₙ, PO2ᵢₙ, T_{in,blood}, PCO2ₒᵤₜ, PO2ₒᵤₜ and T_{out,blood}.

According to some embodiments, the control computer is configured to:
estimate or receive a measurement of a haemoglobin content (Hb) of blood in the bloodstream through of the oxygenator, and
determine the difference ΔCCO2_{blood} between CCO2ᵢₙ and CCO2ₒᵤₜ based on PCO2ᵢₙ, PO2ᵢₙ, T_{in,blood}, PCO2ₒᵤₜ, PO2ₒᵤₜ, T_{out,blood}, and Hb.

According to some embodiments, the control computer is configured to:
calculate a net CO2 exchange (\dot{V}CO2ₙₑₜ) over the membrane based on ΔCCO2_{blood}, and
utilize \dot{V}CO2ₙₑₜ as the measure of CO2 removal.

According to some embodiments, the control computer is configured to:
receive a target value for the measure of CO2 removal, and
automatically regulate the addition of CO2 to the sweep gas flow so as to reach and/or maintain the target value.

According to some embodiments, the device is connected to a patient who is also connected to a mechanical ventilator for mechanically ventilating the patient through the supply of breathing gas to the lungs of the patient, wherein the target value is set to zero in order to evaluate a ventilatory treatment provided by the mechanical ventilator and/or a lung function of the patient.

It is another object of the present disclosure to present a method, a computer program and a system for quantification of CO2 removal in an oxygenator for extracorporeal blood gas exchange, which method, computer program and system is advantageously used to further improve the control of addition of CO2 to an the oxygenator sweep gas flow.

This and other objects, which will become apparent in view of the detailed description following hereinafter, are achieved in accordance with a second aspect of the present disclosure by a method, a computer program and a system as set forth below.

According to this second aspect of the disclosure, there is provided a method for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator. The method comprises the steps of:
measuring a pre-oxygenator fraction of CO2 (FCO2ᵢₙ) in the sweep gas flow upstream of the oxygenator,
measuring a pre-oxygenator sweep gas flow rate (\dot{V}ᵢₙ) of the sweep gas flow upstream of the oxygenator,
measuring a post-oxygenator fraction of CO2 (FCO2ₒᵤₜ) in the sweep gas flow downstream of the oxygenator,
measuring a post-oxygenator sweep gas flow rate (\dot{V}ₒᵤₜ) of the sweep gas flow downstream of the oxygenator, and
calculating a net CO2 exchange (\dot{V}CO2ₙₑₜ) over the membrane based on measured FCO2ᵢₙ, \dot{V}ᵢₙ, FCO2ₒᵤₜ and \dot{V}ₒᵤₜ.

An effect of calculating the net CO2 exchange, \dot{V}CO2ₙₑₜ, over the membrane is that a metric indicating actual CO2 removal in ml/min over the membrane can be presented to an operator of the device, or be used as a control parameter in manual, semi-automatic or automatic control of the sweep gas flow rate and/or an addition of CO2 to the sweep gas flow to meet a set target value for the net CO2 exchange. The proposed technique is advantageous in that it presents a purely gas based approach for determining actual CO2 removal by the oxygenator, without the need for blood gas analysis.

According to some embodiments, the method comprises the steps of:
adding CO2 to the sweep gas flow upstream of the oxygenator to control a degree of CO2 removal from the bloodstream by the oxygenator, and
utilizing \dot{V}CO2ₙₑₜ as a measure of CO2 removal for improved regulation of the CO2 addition to the sweep gas flow.

By utilizing the actual net CO2 exchange for regulation of CO2 addition to the sweep gas flow, an improved and more intuitive regulation of CO2 addition is achieved. In contrast to solutions where CO2 addition is controlled directly based on a difference in measured fractions of CO2 upstream and downstream of the oxygenator (or a difference between other surrogate parameters indicative of CO2 exchange over the membrane), the calculation of \dot{V}CO2ₙₑₜ allows the actual effect of adjustments in CO2 addition to be visualized in terms of a volume of CO2 removal per time unit. Furthermore, in contrast to solutions according to prior art, it allows a non-zero target value for net CO2 removal to be set by the operator, whereby the CO2 addition can be manually, semi-automatically or automatically controlled to make the calculated \dot{V}CO2ₙₑₜ value correspond to the set target value.

According to some embodiments, the step of utilizing the measure of CO2 removal for improved regulation of the addition of CO2 to the sweep gas flow comprises:
presenting the measure of CO2 removal to an operator of the device as decision support in manual adjustment of the addition of CO2 to the sweep gas flow, and/or
presenting, to the operator, a recommendation for adjustment of the addition of CO2 to the sweep gas flow, based on the measure of CO2 removal and a set target for CO2 removal by the oxygenator, and/or
automatically regulating the addition of CO2 to the sweep gas flow based on the measure of CO2 removal and a set target for CO2 removal by the oxygenator.

According to some embodiments, the method comprises the steps of:
measuring or estimating a pre-oxygenator fraction of at least one additional gas in the sweep gas flow upstream of the oxygenator, the at least one additional gas being one or more of water vapour (H2O), oxygen (O2), nitrogen gas (N2), and an anaesthetic agent, and/or
measuring or estimating a post-oxygenator fraction of the at least one additional gas in the sweep gas flow downstream of the oxygenator, and
calculating a compensated pre-oxygenator sweep flow rate (\dot{V}_{in,comp}) based on \dot{V}ᵢₙ, FCO2ᵢₙ and the pre-oxygenator fraction of the at least one additional gas, and/or
calculating a compensated post-oxygenator sweep flow rate (\dot{V}_{out,comp}) based on \dot{V}ₒᵤₜ, FCO2ₒᵤₜ and the post-oxygenator fraction of the at least one additional gas, and
calculating \dot{V}CO2ₙₑₜ based on at least one of \dot{V}_{in,comp} and \dot{V}_{out,comp}.

By taking the composition of the sweep gas into account and compensating one or both of the pre- and post-oxygenator sweep flow rate measurements \dot{V}ᵢₙ and \dot{V}ₒᵤₜ based on a measured fraction of at least one additional gas in the sweep gas flow upstream and/or downstream the oxygenator, a more accurate value of \dot{V}CO2ₙₑₜ can be obtained. The different and altering compositions of the sweep gas flow upstream and downstream of the oxygenator typically introduce errors in the flow measurements due to characteristics and calibration-related parameters of the flow sensors used for obtaining the measurements. By calculating compensated sweep flow rates \dot{V}_{in,comp} and \dot{V}_{out,comp}, such errors can be eliminated or at least substantially mitigated.

According to some embodiments, the at least one additional gas comprises water vapour.

The high concentration of water vapour in the sweep gas flow downstream of the oxygenator causes several problems when it comes to flow and gas concentration measurements and comparisons in membrane oxygenators. By properly measuring or estimating the fraction of water vapour in the sweep gas flow, gas composition measurements and sweep gas flow rate measurements can be compensated based on the fraction of water vapour in the sweep gas flow upstream and/or downstream of the oxygenator. In particular for measurements of the sweep gas flow rate downstream of the oxygenator where humidity is high, the fraction of water vapour in the gas measured upon needs to be taken into account for more precise flow rate determination.

According to some embodiments, the method comprises the steps of:
calculating a compensated pre-oxygenator fraction of CO2 (FCO2_{in,comp}) representing an estimate of a fraction of CO2 at a point of measurement of \dot{V}ᵢₙ, based on FCO2ᵢₙ and an estimated addition or removal of water vapour (ΔFH2Oᵢₙ) to or from the sweep gas between the point of measurement of \dot{V}ᵢₙ and a point of measurement of FCO2ᵢₙ, and/or
calculating a compensated post-oxygenator fraction of CO2 (FCO2_{out,comp}) representing an estimate of a fraction of CO2 at a point of measurement of \dot{V}ₒᵤₜ, based on FCO2ₒᵤₜ and an estimated addition or removal of water vapour (ΔFH2Oₒᵤₜ) to or from the sweep gas between the point of measurement of \dot{V}ₒᵤₜ and a point of measurement of FCO2ₒᵤₜ, and
calculating \dot{V}CO2ₙₑₜ based on at least one of FCO2_{in,comp} and FCO2_{out,comp}.

For precise calculation of \dot{V}CO2ₙₑₜ, not only the measured flow rates but also the measured fractions of CO2 may need to be compensated in order to accurately reflect the fractions of CO2 in the sweep gas flow measured by the flow sensors. In particular in a situation where a sidestream gas analyser is used for sampling sweep gas from a sweep gas outlet line of the oxygenator, the fraction of CO2 in the sweep gas samples measured upon may deviate substantially from the fraction of CO2 in the sweep gas outlet line due to removal of water vapour from the gas samples to be measured upon. For example, it may be desired to remove water vapour from the gas samples prior to FCO2 measurements in order to avoid condensation of water vapour in the gas analyser. The difference ΔFH2Oₒᵤₜ in fraction of H2O between sweep gas in the outlet line (where post-oxygenator flow measurements take place) and the sweep gas samples measured upon by the sidestream gas analyser introduces a discrepancy between measured FCO2ₒᵤₜ and the actual fraction of CO2 at the point of flow measurements. This discrepancy can be eliminated or at least substantially mitigated by calculating a compensated FCO2ₒᵤₜ, comp based on FCO2ₒᵤₜ and an estimated addition or removal of water vapour, ΔFH2Oₒᵤₜ, to or from the sweep gas between the point of measurement of \dot{V}ₒᵤₜ and a point of measurement of FCO2ₒᵤₜ. The same applies on the upstream-side of the oxygenator although the difference ΔFH2Oᵢₙ in fraction of H2O between sweep gas in the sweep gas inlet line of the oxygenator and the sweep gas samples measured upon by the sidestream gas analyser normally is considerably smaller than on the downstream-side of the oxygenator due to the relatively dry pre-oxygenator sweep gas flow.

For example, in some embodiments, the estimated addition or removal of water vapour, ΔFH2Oₒᵤₜ, to or from the sweep gas between a point of measurement of \dot{V}ₒᵤₜ in the sweep gas outlet line and a point of measurement of FCO2ₒᵤₜ in a sidestream gas analyser may be calculated based on a measured post-oxygenator temperature (T_{out,gas}) of the sweep gas flow downstream of the oxygenator, a measured or estimated post-oxygenator relative humidity (RHₒᵤₜ) of the sweep gas flow downstream of the oxygenator, a measured or estimated reference temperature (T_{ref}) at or close to the point of measurement of FCO2ₒᵤₜ, and a measured or estimated reference relative humidity (RH_{ref}) at or close to the point of measurement of FCO2ₒᵤₜ. Likewise, the estimated addition or removal of water vapour, ΔFH2Oᵢₙ, to or from the sweep gas between a point of measurement of \dot{V}ᵢₙ in the sweep gas inlet line and a point of measurement of FCO2ᵢₙ in a sidestream gas analyser may be calculated based on a measured pre-oxygenator temperature (T_{in,gas}) of the sweep gas flow upstream of the oxygenator, a measured or estimated pre-oxygenator relative humidity (RHᵢₙ) of the sweep gas flow upstream of the oxygenator, a measured or estimated reference temperature (T_{ref}) at or close to the point of measurement of FCO2ᵢₙ, and a measured or estimated reference relative humidity (RH_{ref}) at or close to the point of measurement of FCO2ᵢₙ. For example, when removing water vapour from the sampled sweep gas by conditioning the samples using, e.g., a Nafion drying tube prior to providing the sweep gas samples to the sidestream analyser for FCO2 measurements, the reference relative humidity, RH_{ref}, typically corresponds to the ambient RH surrounding the Nafion drying tube. In other examples, when removing water vapour from the sampled sweep gas by conditioning the samples using, e.g., silica gel prior to providing the sweep gas samples to the sidestream analyser, the reference relative humidity, RH_{ref}, can be assumed to be zero or near zero.

According to some embodiments, \dot{V}_{in,comp} is calculated based on FCO2_{in,comp} and the pre-oxygenator fraction of the at least one additional gas. According to some embodiments, \dot{V}_{out,comp} is calculated based on FCO2ₒᵤₜ, comp and the post-oxygenator fraction of the at least one additional gas.

By calculating the compensated sweep gas flow rates from the compensated fractions of CO2 and, optionally, from compensated measurements of fractions of additional gas, instead of relying on the measured fractions, an even higher accuracy in the sweep gas flow rate measurements and hence in the determination of \dot{V}CO2ₙₑₜ can be obtained.

According to some embodiments, the method comprises the steps of:
receiving a target value for the measure of CO2 removal, and
automatically regulating the addition of CO2 to the sweep gas flow so as to reach and/or maintain the target value for the measure of CO2 removal.

According to some embodiments, the device for extracorporeal blood gas exchange is connected to a patient who is also connected to a mechanical ventilator for mechanically ventilating the patient through the supply of breathing gas to the lungs of the patient, wherein the target value is selected to facilitate evaluation of a ventilatory treatment provided by the mechanical ventilator and/or a lung function of the patient, e.g., by selecting a target value corresponding to zero or near zero CO2 removal.

This facilitates efficient combinatory treatment of the patient by the oxygenator and the ventilator since the quantification of net CO2 removal by the oxygenator allows a clinician to better understand the effects of the ongoing treatment and facilitates diagnosis of the current lung status of the patient. Furthermore, it allows a so called weaning test of the patient to be accurately and automatically performed since the ventilatory treatment provided by the ventilator and/or the lung function of the patient can be reliably evaluated by setting a target value for net CO2 removal by the oxygenator to a zero or near zero value.

The step of measuring the post-oxygenator sweep gas flow rate is advantageously performed by measuring the post-oxygenator sweep gas flow rate as a flow rate of a whole effluent flow of sweep gas leaving the oxygenator. Measuring the flow rate of the whole effluent flow of sweep gas is important for precise calculation of \dot{V}CO2ₙₑₜ.

In most oxygenators, sweep gas is discharged to atmosphere after having passed through the oxygenator. Some oxygenators have more than one outlet for discharge of sweep gas, e.g., as a precautionary measure should one or more oxygenator outlets be occluded during operation. In such a scenario, in order to be able to measure the flow rate of the whole effluent flow of sweep gas leaving the oxygenator, it may be desired to prevent sweep gas from leaving the oxygenator via all but one of the plurality of outlets. Consequently, according to some embodiments in which the oxygenator comprises more than one outlet for sweep gas, the method may comprise the steps of preventing the sweep gas to pass through all but one outlet of the oxygenator, and measuring the post-oxygenator sweep gas flow rate as the flow rate of the sweep gas flowing through said one outlet. Prevention of sweep gas flow through one or more additional outlets of the oxygenator can be achieved by temporarily or permanently occluding the one or more additional outlets, e.g., by plugging the one or more additional outlets using silicon plugs or the like.

According to the second aspect of the disclosure, there is also provided a computer program for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator. The computer program comprises computer-readable instructions which, when executed by a control computer, cause the above described method to be performed.

According to the second aspect of the disclosure, there is also provided a computer program product comprising a non-transitory memory hardware device storing a computer program for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator. The computer program comprising computer-readable instructions which, when executed by a control computer, cause the above described method to be performed.

According to the second aspect of the disclosure, there is also provided a system for controlling CO2 removal in a device for extracorporeal blood gas exchange, wherein the device comprises an oxygenator including a membrane acting as a gas-liquid barrier enabling CO2 transfer between a bloodstream and a sweep gas flow through the oxygenator. The system further comprises at least one control computer configured to:
receive a measurement of a pre-oxygenator fraction of CO2 (FCO2ᵢₙ) in the sweep gas flow upstream of the oxygenator,
receive a measurement of a pre-oxygenator sweep gas flow rate of the sweep gas flow upstream of the oxygenator,
receive a measurement of a post-oxygenator fraction of CO2 (FCO2ₒᵤₜ) in the sweep gas flow downstream of the oxygenator,
receive a measurement of a post-oxygenator sweep gas flow rate of the sweep gas flow downstream of the oxygenator, and
calculate a net CO2 exchange (\dot{V}CO2ₙₑₜ) over the membrane based on measured FCO2ᵢₙ, \dot{V}ᵢₙ, FCO2ₒᵤₜ and \dot{V}ₒᵤₜ.

According to some embodiments, the system comprises a gas regulator for adding CO2 to the sweep gas flow upstream of the oxygenator in order to reduce a degree of CO2 removal from the bloodstream by the oxygenator, wherein the control computer is configured to utilize \dot{V}CO2ₙₑₜ as a measure of CO2 removal for improved regulation of the CO2 addition to the sweep gas flow.

According to some embodiments, the control computer is configured to utilize the measure of CO2 removal for improved regulation of the addition of CO2 to the sweep gas flow by:
causing the measure of CO2 removal to be presented to an operator of the device as decision support in manual adjustment of the addition of CO2 to the sweep gas flow, and/or
causing a recommendation for adjustment of the addition of CO2 to the sweep gas flow to be presented to an operator of the device, based on the measure of CO2 removal and a set target for CO2 removal by the oxygenator, and/or
automatically regulating the addition of CO2 to the sweep gas flow based on the measure of CO2 removal.

According to some embodiments, the control computer is configured to:
estimate or receive a measurement of a pre-oxygenator fraction of at least one additional gas in the sweep gas flow upstream of the oxygenator, the at least one additional gas being one or more of water vapour (H2O), oxygen (O2), nitrogen gas (N2), and an anaesthetic agent, and/or
estimate or receive a measurement of a post-oxygenator fraction of the at least one additional gas in the sweep gas flow downstream of the oxygenator, and
calculate a compensated pre-oxygenator sweep flow rate (\dot{V}_{in,comp}) based on \dot{V}ᵢₙ, FCO2ᵢₙ and the pre-oxygenator fraction of the at least one additional gas, and/or
calculate a compensated post-oxygenator sweep flow rate (\dot{V}_{out,comp}) based on \dot{V}ₒᵤₜ, FCO2ₒᵤₜ and the post-oxygenator fraction of the at least one additional gas, and
calculate \dot{V}CO2ₙₑₜ based on at least one of \dot{V}_{in,comp} and \dot{V}_{out,comp}.

According to some embodiments, the at least one additional gas comprises water vapour.

According to some embodiments, the control computer is configured to:
calculate a compensated pre-oxygenator fraction of CO2 (FCO2_{in,comp}) representing an estimate of a fraction of CO2 at a point of measurement (P1) of \dot{V}ᵢₙ, based on FCO2ᵢₙ and an estimated addition or removal of water vapour (ΔFH2Oᵢₙ) to or from the sweep gas between the point of measurement (P1) of \dot{V}ᵢₙ and a point of measurement (P2) of FCO2ᵢₙ, and/or
calculate a compensated post-oxygenator fraction of CO2 (FCO2_{out,comp}) representing an estimate of a fraction of CO2 at a point of measurement (P3) of \dot{V}ₒᵤₜ, based on FCO2ₒᵤₜ and an estimated addition or removal of water vapour (ΔFH2Oₒᵤₜ) to or from the sweep gas between the point of measurement (P3) of \dot{V}ₒᵤₜ and a point of measurement (P4) of FCO2ₒᵤₜ, and
calculate \dot{V}CO2ₙₑₜ based on at least one of FCO2_{in,comp} and FCO2_{out,comp}.

According to some embodiments, the control computer is configured to calculate an estimated addition or removal of water vapour, ΔFH2Oₒᵤₜ, to or from the sweep gas between a point of measurement of \dot{V}ₒᵤₜ in the sweep gas outlet line and a point of measurement of FCO2ₒᵤₜ in a sidestream gas analyser based on a measured post-oxygenator temperature (T_{out,gas}) and a measured post-oxygenator relative humidity (RHₒᵤₜ) of the sweep gas flow downstream of the oxygenator, and a measured or estimated reference temperature (T_{ref}) and a measured or estimated reference relative humidity (RH_{ref}) at or close to the point of measurement of FCO2ₒᵤₜ.

Likewise, the control computer may be configured to calculate an estimated addition or removal of water vapour, ΔFH2Oᵢₙ, to or from the sweep gas between a point of measurement of \dot{V}ᵢₙ in the sweep gas inlet line and a point of measurement of FCO2ᵢₙ in a sidestream gas analyser based on a measured pre-oxygenator temperature (T_{in,gas}) and a measured or estimated pre-oxygenator relative humidity (RHᵢₙ) of the sweep gas flow upstream of the oxygenator, and a measured or estimated reference temperature (T_{ref}) and a measured or estimated reference relative humidity (RH_{ref}) at or close to the point of measurement of FCO2ᵢₙ.

According to some embodiments, \dot{V}_{in,comp} is calculated based on FCO2_{in,comp} and the pre-oxygenator fraction of the at least one additional gas. According to some embodiments, \dot{V}_{out,comp} is calculated based on FCO2ₒᵤₜ, comp and the post-oxygenator fraction of the at least one additional gas.

According to some embodiments, the control computer is configured to:
receive a target value for the measure of CO2 removal, and
regulating the addition of CO2 to the sweep gas flow so as to reach and/or maintain the target value for the measure of CO2 removal.

According to some embodiments, the device for extracorporeal blood gas exchange is connected to a patient who is also connected to a mechanical ventilator for mechanically ventilating the patient through the supply of breathing gas to the lungs of the patient, wherein the target value is selected to facilitate evaluation of a ventilatory treatment provided by the mechanical ventilator and/or a lung function of the patient, e.g., by selecting a target value corresponding to zero or near zero CO2 removal.

More advantageous features of the method, computer program and system of the first and second aspects of the present disclosure will be described in the detailed description following hereinafter.

### BRIEF DESCRIPTION OF THE DRAWINGS

The present invention will become more fully understood from the detailed description provided hereinafter and the accompanying drawings, which are given by way of non-limiting illustration only. In the different drawings, same reference numerals correspond to the same element.

FIG. 1 illustrates an exemplary non-limiting embodiment of an ECMO-vent system for extracorporeal removal of CO2 from the blood of a patient undergoing mechanical ventilation.

FIG. 2 illustrates an exemplary non-limiting embodiment of an ECMO device of the ECMO-vent system in FIG. 1.

FIGS. 3-5 are flow charts illustrating an exemplary non-limiting embodiment of a method for controlling CO2 removal in and by the ECMO device, according to a first aspect of the disclosure.

FIG. 6 is a flowchart illustrating an exemplary non-limiting embodiment of a method for controlling CO2 removal in and by the ECMO device, according to a second aspect of the disclosure.

### DETAILED DESCRIPTION

The present disclosure relates to the field of extracorporeal blood gas exchange by use of an oxygenator for extracorporeal removal of carbon dioxide (CO2) from the blood of a patient. In particular, the disclosure relates to a method, a computer program and a system for improved control of CO2 removal by the oxygenator through addition of CO2 to a sweep gas flow through the oxygenator.

The invention will be described in the context of a combined system for extracorporeal blood gas exchange via a membrane oxygenator during lung protective ventilation of the patient using a mechanical ventilator. However, it should be appreciated that the proposed control of the oxygenator is not dependent on the ventilator and that the operation of the oxygenator does not require the presence of a ventilator.

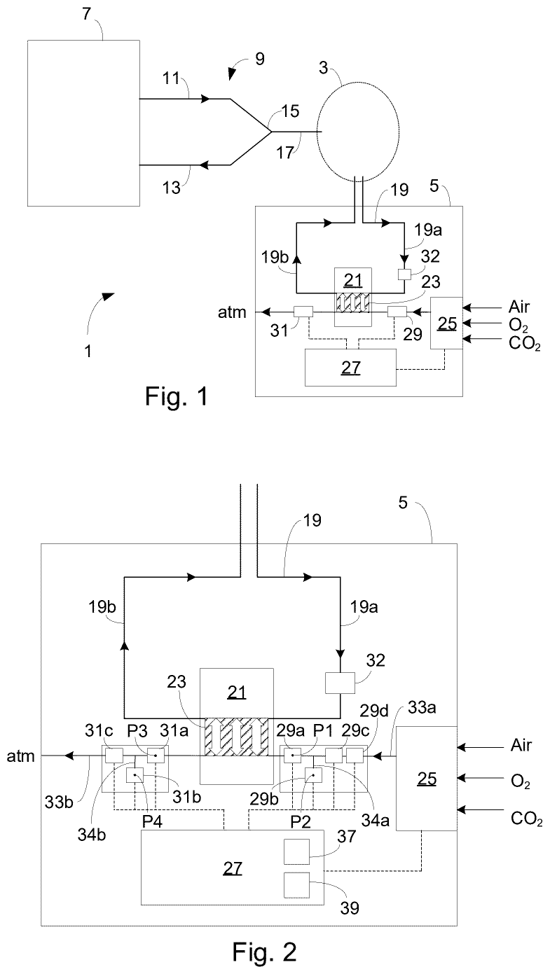

FIG. 1 illustrates a system 1 for combined mechanical ventilation of the lungs of a patient 3 and extracorporeal removal of CO2 from the blood of the patient 3. The system 1 will hereinafter referred to as an ECMO-vent system. ECMO (extracorporeal membrane oxygenation) is one of several terms used for extracorporeal blood gas exchange where blood is pumped outside the body of a treated patient to a device, sometimes referred to as a heart-lung machine, which removes CO2 and sends oxygen-enriched blood back to the patient. Other terms that are frequently used in the art for the same or similar treatments are ECLA (extracorporeal lung assist), ECCO2R (extracorporeal CO2 removal), ECLS (extracorporeal life support) and ECGE (extracorporeal membrane gas-exchange), all of which are encompassed by the term ECMO as used herein.

The ECMO-vent system 1 comprises a device 5, hereinafter referred to as an ECMO device, for extracorporeal removal of CO2 from the blood of the patient 3, and a mechanical ventilator 7 for mechanically ventilating the patient 3 through the supply of breathing gas to the lungs of the patient.

The ventilator 7 comprises or is connected to a source of pressurised breathing gas (not shown), which breathing gas is supplied to the patient 3 via a patient circuit 9. In this example, the patient circuit 9 comprises an inspiratory line 11 for conveying a flow of breathing gas to the patient 3, and an expiratory line 13 for conveying a flow of exhalation gas exhaled by the patient away from the patient. The inspiratory line 11 and the expiratory line 13 are connected to each other via a so called Y-piece 15 which, in turn, is connected to the patient 3 via a common line 17.

The ECMO device 5 is configured to provide ECMO treatment to the patient 3 by generating an extracorporeal flow of blood from the patient 3, oxygenating the blood through extracorporeal blood gas exchange in which CO2 is removed from, and oxygen (O2) added to, the extracorporeal blood flow, and returning the oxygen-enriched blood to the patient 3.

To generate the flow of blood to and from the patient 3, the ECMO device 5 may comprise a blood flow generator (not shown), typically in form of one or several roller, turbine and/or centrifugal pumps. The blood flow generator generates a flow of blood through a tubing system forming a blood flow channel 19 of the ECMO device 5, where parts of the channel may be heated to maintain a desired temperature of the blood when returned to the patient 3.

The blood gas exchange, including blood oxygenation and CO2 removal, takes place in a membrane oxygenator 21 of the ECMO device 5, in which an oxygen-containing sweep gas flow interacts with the blood in the blood flow channel 19 via a membrane 23 of the oxygenator 21. The membrane 23 acts as a gas-liquid barrier enabling transfer of CO2 and O2 content between the bloodstream flowing through the oxygenator 21 on a liquid-side of the membrane 23 and the sweep gas flow flowing through the oxygenator 21 on a gas-side of the membrane 23.

The sweep gas flow is generated by a sweep gas generator 25 connected to one or more sweep gas sources, typically including one or both of an oxygen source and a source of compressed air. According to the principles of the present disclosure, the sweep gas generator 25 is further connected to a CO2 source in order to control the degree of CO2 removal over the oxygenator 21 through addition of CO2 to the sweep gas flow. The sweep gas generator 23 is configured to deliver a controllable sweep gas composition to the oxygenator 21 at a controllable sweep gas flow rate.

The composition and, optionally, the flow rate of the sweep gas generated by the sweep gas generator 23 may be automatically controlled by a controller or control computer 27 of the ECMO device 5 based on set target values and sensor data obtained by various sensors 29, 31 of the ECMO device 5. In particular, the control computer 27 of the ECMO device 5 may be configured to automatically control an addition of CO2 to a sweep gas flow comprising any or both of oxygen and air, based on a set target for a measure of CO2 removal by the oxygenator 21.

Hereinafter, the sweep gas flow upstream of the oxygenator 21 (i.e., before the oxygenator from the sweep gas' point of view) will be referred to as an input sweep gas flow or a pre-oxygenator sweep gas flow, and the sweep gas flow downstream of the oxygenator 21 (i.e., after the oxygenator from the sweep gas' point of view) will be referred to as an output sweep gas flow or a post-oxygenator sweep gas flow. The input sweep gas flow flows from the sweep gas generator 25 to the oxygenator 21 via a sweep gas inlet line 33a of the ECMO device 5, and the output sweep gas flow flows from the oxygenator 21 to atmosphere or an evacuation or recirculation system via a sweep gas outlet line 33b. In most configurations, ECMO systems are open systems, meaning that the post oxygenator sweep gas flow is allowed to escape into the ambient. In some cases, especially when anesthetic agents are added to the sweep gas flow, a closed or semi closed (sweep) gas control system can be envisioned, similar to gas control systems often used in anesthesia machines.

Likewise, the bloodstream upstream of the oxygenator 21 (i.e., before the oxygenator from the bloodstream's point of view) may hereinafter be referred to as an input bloodstream or pre-oxygenator bloodstream, and the bloodstream downstream of the oxygenator 21 (i.e., after the oxygenator from the bloodstream's point of view) may be referred to as an output bloodstream or post-oxygenator bloodstream. The input bloodstream flows from the patient 3 to the oxygenator 21 via a bloodstream inlet line 19a of the ECMO device 5, and the output bloodstream flows from the oxygenator 21 and back to the patient 3 via a bloodstream outlet line 19b of the ECMO device 5.

With reference now made to FIG. 2, the sensors 29, 31 of the ECMO device 5 may comprise:
a pre-oxygenator flow rate sensor 29a for measuring a flow rate of the input sweep gas flow, \dot{V}ᵢₙ. The pre-oxygenator flow rate sensor 29a is a mainstream flow sensor, meaning that it is configured to measure the flow rate of the sweep gas flowing in the sweep gas inlet line 33a. The pre-oxygenator flow measurements obtained by the pre-oxygenator flow rate sensor 29a take place at a pre-oxygenator point of flow measurement denoted P1 in the sweep gas inlet line 33a.
a pre-oxygenator gas analyser 29b for measuring a fraction of at least CO2 in the input sweep gas flow, FCO2ᵢₙ. The pre-oxygenator gas analyser 29b may also be configured to measure a fraction of one or more additional gases selected from the group consisting of oxygen (O2), nitrogen gas (N2), and anaesthetic agents. In this exemplary embodiment, the pre-oxygenator gas analyser 29b is a so called sidestream gas analyser that is configured to withdraw sweep gas samples from the sweep gas inlet line 33a, and measure the fraction of CO2 and, optionally, the fraction of the at least one additional gas in the sweep gas samples at a pre-oxygenator point of CO2 measurement denoted P2. The pre-oxygenator point of CO2 measurement P2 is separated in distance from the point of pre-oxygenator sweep gas flow rate measurements, P1, at least by the length of a pre-oxygenator sample line 34a. In other embodiments, the pre-oxygenator gas analyser 29b may be a so called mainstream gas analyser that is configured to measure the fraction of CO2 and, optionally, the fraction of the at least one additional gas within the sweep gas inlet line 33a. In some embodiments, the pre-oxygenator gas analyser comprises at least a CO2 sensor and an O2 sensor for measuring a fraction of CO2 and O2, respectively, in the sweep gas samples. In some embodiments, the CO2 sensor is a non-dispersive infrared (NDIR) CO2 sensor. In some embodiments, the O2 sensor is a paramagnetic or electrochemical O2 sensor.
a pre-oxygenator temperature sensor 29c for measuring a temperature of the input sweep gas, T_{in,gas}.
a pressure sensor 29d for measuring a sweep gas circuit pressure, P_{gas}, substantially corresponding to the sweep gas pressure in the gas inlet line 33a.
a post-oxygenator flow rate sensor 31a for measuring a flow rate of the output sweep gas flow, \dot{V}ₒᵤₜ. The post-oxygenator flow rate sensor 31a is a mainstream flow sensor, meaning that it is configured to measure the flow rate of the sweep gas flowing in the sweep gas outlet line 33b. The post-oxygenator flow measurements obtained by the post-oxygenator flow rate sensor 31a take place at a post-oxygenator point of flow measurement denoted P3 in the sweep gas outlet line 33b.
a post-oxygenator gas analyser 31b for measuring a fraction of at least CO2 in the output sweep gas flow, FCO2ₒᵤₜ. The post-oxygenator gas analyser 31b may also be configured to measure a fraction of one or more additional gases selected from the group consisting of oxygen (O2), nitrogen gas (N2), and anaesthetic agents. In this exemplary embodiment, the post-oxygenator gas analyser 31b is a so called sidestream gas analyser that is configured to withdraw sweep gas samples from the sweep gas outlet line 33b, and measure the fraction of CO2 and, optionally, the fraction of the at least one additional gas in the sweep gas samples at a post-oxygenator point of CO2 measurement denoted P4. The post-oxygenator point of CO2 measurement P4 is separated in distance from the point of post-oxygenator sweep gas flow rate measurements, P3, at least by the length of a post-oxygenator sample line 34b. In other embodiments, the post-oxygenator gas analyser 31b may be a so called mainstream gas analyser that is configured to measure the fraction of CO2 and, optionally, the fraction of the at least one additional gas within the sweep gas outlet line 33b. In some embodiments, the post-oxygenator gas analyser comprises at least a CO2 sensor and an O2 sensor for measuring a fraction of CO2 and O2, respectively, in the sweep gas samples. In some embodiments, the CO2 sensor is an IR spectrometer for IR spectroscopy, such as IR absorption spectroscopy. In some embodiments, the O2 sensor is a paramagnetic O2 sensor.
a post-oxygenator temperature sensor 31c for measuring a temperature of the output sweep gas.

In some embodiments, the ECMO device 5 may further comprise or be connected to a pre-oxygenator blood gas analyser 32 for measuring a partial pressure of at least CO2 in the input bloodstream, PCO2ᵢₙ. The pre-oxygenator blood gas analyser 32 may also be configured to measure a partial pressure of O2 in the input bloodstream, PO2ᵢₙ. The pre-oxygenator blood gas analyser 32 may also be configured to measure a haemoglobin content of the input bloodstream, Hbᵢₙ. In some embodiments, the blood gas analyser 32 is not incorporated into the ECMO device 5 but arranged to form part of another medical device that is connected to the ECMO device 5 in order for the ECMO device 5 to receive measurements obtained by the blood gas analyser. For example, the blood gas analyser may form part of a stand-alone blood gas analyser unit, often referred to as a BGA, commonly used for intermittent blood gas analysis during ECMO treatments.

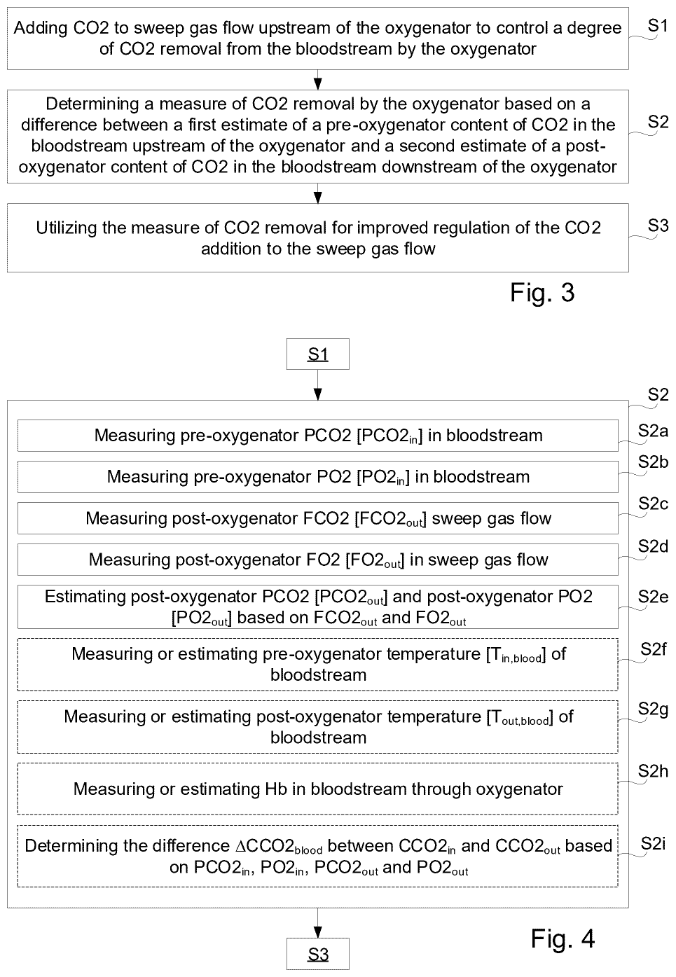

With simultaneous reference to previous drawings, some functions and features of the ECMO-vent system 1 will now be described with reference to the flowcharts shown in FIGS. 3-6, which flowcharts illustrate methods for controlling CO2 removal in and by the ECMO device 5. Unless stated otherwise, each method is a computer-implemented method that is performed by the ECMO device 5 upon execution of a computer program by at least one processor 37 of the control computer 27. The computer program(s) comprise computer-readable instructions that may be stored in a storage medium of the ECMO-vent system 1, such as a non-transitory hardware memory device 39 of the control computer 27.

FIG. 3 is a flowchart illustrating a method for controlling CO2 removal in and by the ECMO device according to a first aspect of the present disclosure.

In a first step, S1, CO2 is added to the sweep gas flow upstream of the oxygenator 25 in order to control a degree of CO2 removal from the bloodstream by the oxygenator 21. CO2 is added to the sweep gas flow via the manually, semi-automatically or automatically controlled sweep gas regulator 25.

In a second step, S2, a measure of CO2 removal by the oxygenator 21 is determined based on a difference (ΔCCO2_{blood}) between a measure of a pre-oxygenator content of CO2 (CCO2ᵢₙ) in the bloodstream upstream of the oxygenator 21 and an estimate of a post-oxygenator content of CO2 (CCO2ₒᵤₜ) in the bloodstream downstream of the oxygenator 21. The determination is made by the control computer 27 based on sensor data obtained by the sensors 29a-29d, 31a-31c and 32.

In a third step, S3, the measure of CO2 removal is utilized for improved regulation of the CO2 addition to the sweep gas flow.

In some embodiments, the measure of CO2 removal is determined from pre-oxygenator measurements of partial pressures of CO2 (PCO2ᵢₙ) and 02 (PO2ᵢₙ) in the input bloodstream, e.g., obtained by the blood gas analyser 32, and post-oxygenator measurements of fractions of CO2 (FCO2ₒᵤₜ) and O2 (FO2ₒᵤₜ) in the output sweep gas flow, e.g., obtained by the post-oxygenator gas analyser 31b.

The measure of the pre-oxygenator content of CO2, CCO2ᵢₙ, in the input bloodstream may be expressed as a function of a pre-oxygenator partial pressure of CO2 (PCO2ᵢₙ) of the bloodstream, a pre-oxygenator partial pressure of O2 (PO2ᵢₙ) of the bloodstream, a pre-oxygenator temperature (T_{in,blood}) of the bloodstream, and a pre-oxygenator haemoglobin concentration (Hbᵢₙ) of the bloodstream. Likewise, the estimate of the post-oxygenator content of CO2, CCO2ₒᵤₜ, in the output bloodstream may be expressed as a function of a post-oxygenator partial pressure of CO2 (PCO2ₒᵤₜ) of the bloodstream, a post-oxygenator partial pressure of O2 (PO2ₒᵤₜ) of the bloodstream, a post-oxygenator temperature of the bloodstream (T_{out,blood}), and a post-oxygenator haemoglobin concentration (Hbₒᵤₜ) of the bloodstream. PCO2ᵢₙ and PO2ᵢₙ may be measured by the pre-oxygenator blood gas analyser 32, whereas PCO2ₒᵤₜ and PO2ₒᵤₜ can be assumed to substantially correspond to measured post-oxygenator fractions of CO2 (FCO2ₒᵤₜ) and O2 (FO2ₒᵤₜ) in the outlet sweep gas flow. Since the Hb concentration of the bloodstream can be assumed to be constant, Hbᵢₙ and Hbₒᵤₜ are cancelled out and the difference between the estimates of CCO2ᵢₙ and CCO2ₒᵤₜ can be calculated from FCO2ᵢₙ, FO2ᵢₙ, FCO2ₒᵤₜ, FO2ₒᵤₜ, T_{in,blood} and T_{out,blood}.

FIG. 4 is a flowchart illustrating a non-limiting example of how the determination of the measure of CO2 removal in step S2 in FIG. 3 can be achieved in more detail.

As illustrated in FIG. 4, step S2 may comprise the following sub-steps:
S2a) measuring a pre-oxygenator partial pressure of CO2 (PCO2ᵢₙ) in the bloodstream upstream of the oxygenator 21, e.g., by means of the pre-oxygenator blood gas analyser 32,
S2b) measuring a pre-oxygenator partial pressure of O2 (PO2ᵢₙ) in the bloodstream upstream of the oxygenator 21, e.g., by means of the pre-oxygenator blood gas analyser 32,
S2c) measuring a post-oxygenator fraction of CO2 (FCO2ₒᵤₜ) in the sweep gas flow downstream of the oxygenator 21, e.g., by means of the post-oxygenator gas analyser 31b,
S2d) measuring a post-oxygenator fraction of O2 (FO2ₒᵤₜ) in the sweep gas flow downstream of the oxygenator 21, e.g., by means of the post-oxygenator gas analyser 31b,
S2e) estimating a post-oxygenator partial pressure of CO2 (PCO2ₒᵤₜ) and a post-oxygenator partial pressure of O2 (PO2ₒᵤₜ) in the bloodstream downstream of the oxygenator 21 based on FCO2ₒᵤₜ and FO2ₒᵤₜ,
S2f) measuring or estimating a pre-oxygenator temperature (T_{in,blood}) of blood in the bloodstream upstream of the oxygenator 21. T_{in,blood} may be measured with a pre-oxygenator blood temperature sensor (not shown), or it may be estimated e.g. based on an assumed and/or measured temperature of the patient 3, a temperature of the sweep gas flow, a length of tubing of the blood flow channel 19, and/or an effect of a heater (not shown) for heating blood in the blood flow channel,
S2g) measuring or estimating a post-oxygenator temperature (T_{out,blood}) of blood in the bloodstream downstream of the oxygenator 21. T_{out,blood} may be measured with a post-oxygenator blood temperature sensor (not shown), or it may be estimated e.g. based on an assumed and/or measured temperature of the patient 3, a temperature of the sweep gas flow, a length of tubing of the blood flow channel 19, and/or an effect of a heater (not shown) for heating blood in the blood flow channel.
S2h) measuring or estimating a haemoglobin content (Hb) of blood in the bloodstream through of the oxygenator 21, e.g., by means of the pre-oxygenator blood gas analyser 32, and
S2i) determining the difference ΔCCO2_{blood} between CCO2ᵢₙ and CCO2ₒᵤₜ from the measured and/or estimated quantities. PCO2ᵢₙ, PO2ᵢₙ, T_{in,blood}, PCO2ₒᵤₜ, PO2ₒᵤₜ, T_{out,blood}, and Hb.

As illustrated by dashed lines in FIG. 4, the steps of measuring or estimating T_{in,blood}, T_{out,blood} and Hb are optional. Since, for some cases and some oxygenator configurations, T_{in,blood} can be assumed to substantially correspond to T_{out,blood}, and since Hb is constant upstream and downstream of the oxygenator 21, the difference ΔCCO2_{blood} between CCO2ᵢₙ and CCO2ₒᵤₜ can be approximated from PCO2ᵢₙ, PO2ᵢₙ, PCO2ₒᵤₜ, and PO2ₒᵤₜ alone. However, to further improve accuracy in the determination, T_{in,blood} and T_{out,blood} may be taken into account. By determining and utilising Hb in the determination of the difference ΔCCO2_{blood}, the difference can be quantified and an actual net CO2 exchange, \dot{V}CO2ₙₑₜ, can be calculated and used as a measure of CO2 exchange over the oxygenator 21.

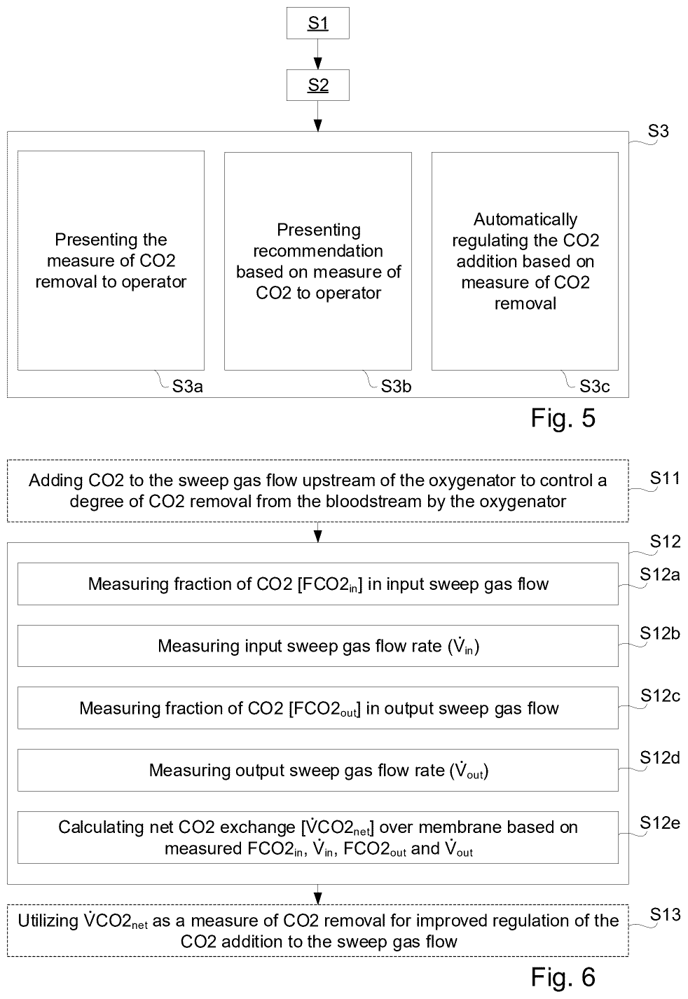

FIG. 5 is a flowchart illustrating some non-limiting examples of how the determined measure of CO2 removal can be utilized in step S3 in FIG. 3 in order to improve regulation of the CO2 addition to the sweep gas flow.

As illustrated in FIG. 5, step S3 may comprise one or more of the following steps:
S3a) presenting the measure of CO2 removal to an operator of the ECMO device 5 as decision support in manual adjustment of the addition of CO2 to the sweep gas flow. The measure of CO2 removal may, for example, be presented on a display comprised in or connected to the ECMO device 5.
S3b) presenting, to the operator of the ECMO device 5, a recommendation for adjustment of the addition of CO2 to the sweep gas flow, based on the measure of CO2 removal and a set target for CO2 removal by the oxygenator. For example, the recommendation may be presented on a display comprised in or connected to the ECMO device 5. The recommendation may, for example, be a recommendation to increase or decrease the addition of CO2 to the sweep gas flow, e.g., by manually increasing or decreasing a set value for the fraction of CO2 (FCO2ᵢₙ) in the sweep gas flow upstream of the oxygenator 21.
S3c) automatically regulating the addition of CO2 to the sweep gas flow based on the measure of CO2 removal and a set target for CO2 removal. For example, if the set target is zero CO2 removal, the control computer 27 may be configured to control the sweep gas regulator 25 to regulate the fraction of CO2 in the inlet sweep gas flow so as to reach and/or maintain zero CO2 removal.

FIG. 6 is a flowchart illustrating a method for controlling CO2 removal in and by the ECMO device 5 according to a second aspect of the present disclosure.

In a first optional step, S11, CO2 is added to the sweep gas flow upstream of the oxygenator 21 to control a degree of CO2 removal from the bloodstream of the patient 3 by the oxygenator 21.

In a second step, S12, a net CO2 exchange (\dot{V}CO2ₙₑₜ) over the membrane 23 of the oxygenator 21 is calculated.

Step S12 comprises the following sub-steps:
S12a) measuring a pre-oxygenator fraction of CO2 (FCO2ᵢₙ) in the sweep gas flow upstream of the oxygenator 21, e.g., by means of the pre-oxygenator gas analyser 29b;
S12b) measuring a pre-oxygenator sweep gas flow rate (\dot{V}ᵢₙ) of the sweep gas flow upstream of the oxygenator 21, e.g., by means of the pre-oxygenator flow rate sensor 29a;
S12c) measuring a post-oxygenator fraction of CO2 (FCO2ₒᵤₜ) in the sweep gas flow downstream of the oxygenator 21, e.g., by means of the post-oxygenator gas analyser 31b;
S12d) measuring a post-oxygenator sweep gas flow rate (\dot{V}ₒᵤₜ) of the sweep gas flow downstream of the oxygenator 21, e.g., by means of a post-oxygenator flow rate sensor 31a, and
S12e) calculating \dot{V}CO2ₙₑₜ over the membrane 23 based on measured FCO2ᵢₙ, \dot{V}ᵢₙ, FCO2ₒᵤₜ and \dot{V}ₒᵤₜ.

In a third optional step, S13, \dot{V}CO2ₙₑₜ is utilized as a measure of CO2 removal for improved regulation of the CO2 addition to the sweep gas flow. Step S13 may comprise any of, or any combination of, the steps S3a-S3c illustrated in FIG. 5, using the calculated \dot{V}CO2ₙₑₜ as the measure of CO2 removal.

\dot{V}CO2ₙₑₜ may be calculated as the fraction of CO2 (FCO2ᵢₙ) in the input sweep gas flow times the input sweep gas flow rate (\dot{V}ᵢₙ), minus the fraction of CO2 (FCO2ₒᵤₜ) in the output sweep gas flow times the output sweep gas flow rate (\dot{V}ₒᵤₜ), in accordance with:

$$\dot{V} CO {2}_{net} = ( F CO {2}_{in} * {\dot{V}}_{in} ) - ( F CO {2}_{out} * {\dot{V}}_{out} ) \qquad eq . l$$

Of course, in order to accurately calculate the flow of CO2 going into and out from the oxygenator 21, the terms FCO2ᵢₙ and FCO2ₒᵤₜ should reflect the true fractions of CO2 at the points of measurements of \dot{V}ᵢₙ and \dot{V}ₒᵤₜ. Due to the humid environment (often close to 100% RH) downstream the oxygenator, there is often a need for conditioning (e.g., drying) the sweep gas before it enters the gas analyser, e.g., to prevent condensation of water inside the gas analyser. Therefore, with reference again made to FIG. 2, the ECMO device 5 may comprise a water vapour trap or gas sample conditioner (not shown) for conditioning and especially for drying the sweep gas samples withdrawn from the sweep gas outlet line 33b before the sweep gas samples enter the post-oxygenator gas analyser 31b. The gas sample conditioner may, e.g., comprise a piece of Nafion tubing or silica gel. Due to the removal of water by the gas sample conditioner, the composition of the sweep gas samples measured upon is not the same as the composition of the sweep gas flow in the sweep gas outlet line 33b, where \dot{V}ₒᵤₜ is measured. Therefore, the fraction of CO2, FCO2ₒᵤₜ, measured by the sidestream post-oxygenator gas analyser 31b at the point of measurement P4 will not accurately reflect the fraction of CO2 at the point, P3, of output sweep gas flow rate measurements.

To this end, the method may further comprise the steps of:
calculating a compensated pre-oxygenator fraction of CO2 (FCO2_{in,comp}) representing an estimate of a fraction of CO2 at the point of measurement, P1, of \dot{V}ᵢₙ, based on FCO2ᵢₙ and an estimated addition or removal of water vapour, ΔFH2Oᵢₙ, to or from the sweep gas between the point of measurement, P1, of \dot{V}ᵢₙ and a point of measurement, P2, of FCO2ᵢₙ, and/or
calculating a compensated post-oxygenator fraction of CO2 (FCO2_{out,comp}) representing an estimate of a fraction of CO2 at a point of measurement, P3, of \dot{V}ₒᵤₜ, based on FCO2ₒᵤₜ and an estimated addition or removal of water vapour (ΔFH2Oₒᵤₜ) to or from the sweep gas between the point of measurement, P3, of \dot{V}ₒᵤₜ and a point of measurement, P4, of FCO2ₒᵤₜ, and
calculating \dot{V}CO2ₙₑₜ based on \dot{V}ᵢₙ and \dot{V}ₒᵤₜ, and at least one of FCO2_{in,comp} and FCO2ₒᵤₜ, comp.

For example, a compensated post-oxygenator fraction of CO2 (FCO2_{out,comp}) can be calculated as a function of measured post-oxygenator fraction of CO2 (FCO2ₒᵤₜ) and an estimated addition or removal of water vapour (ΔFH2Oₒᵤₜ) to or from the sweep gas between the point of measurement, P3, of \dot{V}ₒᵤₜ and a point of measurement, P4, of FCO2ₒᵤₜ, and used in the determination of \dot{V}CO2ₙₑₜ according to:

$${F C O2}_{out , comp} = f ( F CO {2}_{out} , Δ F H 2 {O}_{out} ) \qquad eq . 2$$

$$\dot{V} CO {2}_{net} = ( F CO {2}_{in} * {\dot{V}}_{in} ) - ( {F CO2}_{out , comp} * {\dot{V}}_{out} ) \qquad eq . 3$$

The estimated addition or removal of water vapour, ΔFH2Oₒᵤₜ, may, in some embodiments, be calculated based on: a measured post-oxygenator temperature (T_{out,gas}), measured by the post-oxygenator temperature sensor 31c; a measured or estimated post-oxygenator relative humidity (RHₒᵤₜ) of the sweep gas flow downstream of the oxygenator; a measured or estimated reference temperature (T_{ref}) at or close to the point of measurement P4 of FCO2ₒᵤₜ, and; a measured or estimated reference relative humidity (RH_{ref}) at or close to the point of measurement P4 of FCO2ₒᵤₜ. In embodiments where the gas sample conditioner comprises a piece of Nafion tubing, the reference relative humidity, RH_{ref}, can be assumed to correspond to the relative humidity of the air surrounding the Nafion tubing. In embodiments where the gas sample conditioner comprises silica gel, the reference relative humidity, RH_{ref}, can be assumed to be zero or near zero. The post-oxygenator relative humidity, RHₒᵤₜ can in most situations be assumed to be 100% but may, in some embodiments, be measured by a humidity sensor (not shown) of the ECMO device 5, arranged downstream of the oxygenator 21.

Another potential source of error in the calculation of \dot{V}CO2ₙₑₜ is inaccuracy in sweep gas flow rate measurements. The flow sensors 29a and 31a for measuring \dot{V}ᵢₙ and \dot{V}ₒᵤₜ are normally calibrated for a specific gas composition and deviations between an assumed composition and an actual composition of the sweep gas measured upon introduces errors in flow rate determinations. Therefore, the method may further comprise the steps of:
measuring or estimating a pre-oxygenator fraction of at least one additional gas in the sweep gas flow upstream of the oxygenator 21, the at least one additional gas being one or more of water vapour (H2O), O2, nitrogen gas (N2), and an anaesthetic agent, and/or
measuring or estimating a post-oxygenator fraction of the at least one additional gas in the sweep gas flow downstream of the oxygenator, and
calculating a compensated pre-oxygenator sweep flow rate (\dot{V}_{in,comp}) based on \dot{V}ᵢₙ, FCO2ᵢₙ and the pre-oxygenator fraction of the at least one additional gas, and/or
calculating a compensated post-oxygenator sweep flow rate (\dot{V}_{out,comp}) based on \dot{V}ₒᵤₜ, FCO2ₒᵤₜ and the post-oxygenator fraction of the at least one additional gas, and
calculating \dot{V}CO2ₙₑₜ based on FCO2ᵢₙ, FCO2ₒᵤₜ and at least one of \dot{V}_{in,comp} and \dot{V}_{out,comp}.

By taking the composition of the sweep gas into account and compensating the pre-oxygenator and/or the post-oxygenator sweep flow rate measurements \dot{V}ᵢₙ and \dot{V}ₒᵤₜ based on a measured fraction of at least one additional gas in the sweep gas flow upstream and/or downstream the oxygenator, a more accurate value of \dot{V}CO2ₙₑₜ can be obtained.

For example, a compensated post-oxygenator sweep gas flow rate, \dot{V}_{out,comp}, may be calculated as a function of \dot{V}ₒᵤₜ, FCO2ₒᵤₜ and a post-oxygenator fraction of O2, water vapour and/or N2, according to:

$${\dot{V}}_{out , comp} = f ( {\dot{V}}_{out} , {F CO2}_{out} , {F O2}_{out} , F H 2 {O}_{out} , {F N2}_{out} ) , \qquad eq . 4$$

where FCO2ₒᵤₜ and FO2ₒᵤₜ are the fractions of CO2 and O2 measured by the sidestream post-oxygenator gas analyser 31b, FH2Oₒᵤₜ is the fraction of water vapour in the sweep gas flow downstream of the oxygenator, which may be determined from the measured post-oxygenator temperature, T_{out,gas}, of the sweep gas flow downstream of the oxygenator and the measured or estimated post-oxygenator relative humidity, RHₒᵤₜ, of the sweep gas flow downstream of the oxygenator, and FN2ₒᵤₜ is the post-oxygenator fraction of N2 which may be measured by the post-oxygenator gas analyser 31b or be assumed to correspond to the remaining fraction of the post-oxygenator sweep gas flow.

\dot{V}CO2ₙₑₜ may then be calculated as:

$$\dot{V} CO {2}_{net} = ( F CO {2}_{in} * {\dot{V}}_{in} ) - ( F CO {2}_{out , comp} * {\dot{V}}_{out , comp} ) \qquad eq . 5$$

As discussed above, there is often a need for using a gas sample conditioner for drying the gas samples between the sweep gas sampling point in the sweep gas outlet line 33b and the sidestream post-oxygenator gas analyser 31b. The removal or reduction of water vapour content by the gas sample conditioner introduces a deviation between the composition of the sweep gas in the sweep gas outlet line (where \dot{V}ₒᵤₜ is measured) and the sweep gas samples measured upon by the sidestream gas analyser 31b. Therefore, to further improve accuracy in the determination of the compensated post-oxygenator sweep gas flow rate, \dot{V}_{out,comp}, the compensated post-oxygenator fraction of CO2, FCO2_{out,comp}, may be advantageously used instead of the measured post-oxygenator fraction of CO2, FCO2ₒᵤₜ, in the determination of \dot{V}_{out,comp}, in accordance with:

$${\dot{V}}_{out , comp} = f ( {\dot{V}}_{out} , {F CO2}_{out , comp} , {F O2}_{out} , F H 2 {O}_{out} , {F N2}_{out} ) \qquad eq . 6$$

Any fractions of additional gases measured by the post-oxygenator sidestream gas analyser 31b, such as measured fractions of O2 (FO2ₒᵤₜ) and/or measured fractions of N2 (FN2ₒᵤₜ), may also be compensated based on the estimated addition or removal of water vapour, ΔFH2Oₒᵤₜ, to or from the sweep gas between the point of measurement, P3, of \dot{V}ₒᵤₜ and the point of measurement, P4, of the additional gas by the gas analyser. A compensated fraction of the additional gas may be calculated as a function of the measured fraction of the additional gas and the estimated addition or removal of water vapour, ΔFH2Oₒᵤₜ, in accordance with:

$${F O2}_{out , comp} = f ( {F O2}_{out} , Δ F H 2 {O}_{out} ) \qquad eq . 7$$

$${F N2}_{out , comp} = f ( F N {2}_{out} , Δ F H 2 {O}_{out} ) \qquad eq . 8$$

The compensated fractions of the additional gases may then replace the measured fractions of additional gases in eq. 6 to further improve the accuracy in post-oxygenator sweep gas flow rate determination, in accordance with:

$${\dot{V}}_{out , comp} = f ( {\dot{V}}_{out} , F CO {2}_{out , comp} , {F O2}_{out , comp} , F H 2 {O}_{out} , {F N2}_{out , comp} ) \qquad eq . 9$$

A precise measure of \dot{V}CO2ₙₑₜ may then be calculated using eq. 5 with a \dot{V}_{out,comp} value calculated in accordance with eq. 9.

It should be noted that although described herein as techniques for controlling removal of CO2 from the bloodstream of a patient, the techniques of the first and second aspects of the present disclosure could as well be used to control an addition of CO2 to the bloodstream of the patient via the oxygenator 21. This may sometimes be desired to obtain or maintain a desired pH or partial pressure of CO2 (PCO2) in the blood of the patient 3. Consequently, it should be realised that the disclosed methods for controlling CO2 removal in the ECMO device 5 could be generalised to methods for controlling transfer of CO2 to or from the bloodstream of the patient 3, via the oxygenator 21, in order to control addition or removal of CO2 to or from the blood of the patient. The purpose of controlling CO2 transfer may be any of: 1) ensuring sufficient removal of CO2 from the blood of the patient; 2) keeping CO2 removal constant, e.g., in order to evaluate an ongoing respiratory treatment provided by the mechanical ventilator 7 and/or a lung function of the patient 3, and 3) obtaining or maintaining a set or desired post-oxygenator pH and/or PCO2, in particular during adjustments of the sweep gas flow rate.

In applications where CO2 transfer over the oxygenator membrane 23 is controlled to obtain or maintain a set or desired post-oxygenator pH and/or PCO2, the control computer 27 of the ECMO device 5 may be configured to automatically control the inlet sweep gas flow rate and/or the addition of CO2 to the inlet sweep gas flow to obtain or maintain pH and/or PCO2.

1-20. (canceled)

21. A method for controlling carbon dioxide [CO2] removal in a device for extracorporeal blood gas exchange, wherein the device comprises an oxygenator including a membrane acting as a gas-liquid barrier enabling CO2 transfer between a bloodstream and a sweep gas flow through the oxygenator, comprising the steps of:
measuring (S12a) a pre-oxygenator fraction of CO2 [FCO2ᵢₙ] in the sweep gas flow upstream of the oxygenator;
measuring (S12b) a pre-oxygenator sweep gas flow rate (\dot{V}ᵢₙ) of the sweep gas flow upstream of the oxygenator;
measuring (S12c) a post-oxygenator fraction of CO2 [FCO2ₒᵤₜ] in the sweep gas flow downstream of the oxygenator;
measuring (S12d) a post-oxygenator sweep gas flow rate (\dot{V}ₒᵤₜ) of the sweep gas flow downstream of the oxygenator; and
calculating (S12e) a net CO2 exchange [\dot{V}CO2ₙₑₜ] over the membrane based on measured FCO2ᵢₙ, \dot{V}ᵢₙ, FCO2ₒᵤₜ and \dot{V}ₒᵤₜ.

22. The method of claim 21, further comprising the steps of:
adding (S11) CO2 to the sweep gas flow upstream of the oxygenator to control a degree of CO2 removal from the bloodstream by the oxygenator; and
utilizing (S13) \dot{V}CO2ₙₑₜ as a measure of CO2 removal for regulation of the CO2 addition to the sweep gas flow.

23. The method of claim 22, wherein the step of utilizing the measure of CO2 removal for regulation of the addition of CO2 to the sweep gas flow comprises:
presenting (S13a) the measure of CO2 removal to an operator of the device as decision support in manual adjustment of the addition of CO2 to the sweep gas flow; and/or
presenting (S13b), to the operator, a recommendation for adjustment of the addition of CO2 to the sweep gas flow, based on the measure of CO2 removal and a set target for CO2 removal by the oxygenator; and/or
automatically regulating (S13c) the addition of CO2 to the sweep gas flow based on the measure of CO2 removal.

24. The method of claim 21, further comprising the steps of:
measuring or estimating a pre-oxygenator fraction of at least one additional gas in the sweep gas flow upstream of the oxygenator, the at least one additional gas being one or more of water vapour (H2O), oxygen [O2], nitrogen gas [N2], and an anaesthetic agent; and/or
measuring or estimating a post-oxygenator fraction of the at least one additional gas in the sweep gas flow downstream of the oxygenator; and
calculating a compensated pre-oxygenator sweep flow rate [\dot{V}_{in,comp}] based on \dot{V}ᵢₙ, FCO2ᵢₙ and the pre-oxygenator fraction of the at least one additional gas; and/or
calculating a compensated post-oxygenator sweep flow rate [\dot{V}_{out,comp}] based on \dot{V}ₒᵤₜ, FCO2ₒᵤₜ and the post-oxygenator fraction of the at least one additional gas; and
calculating \dot{V}CO2ₙₑₜ based on at least one of \dot{V}_{in,comp} and \dot{V}_{out,comp}.

25. The method of claim 21, further comprising the steps of:
calculating a compensated pre-oxygenator fraction of CO2 [FCO2_{in,comp}] representing an estimate of a fraction of CO2 at a point of measurement (P1) of \dot{V}ᵢₙ, based on FCO2ᵢₙ and an estimated addition or removal of water vapour [ΔFH2Oᵢₙ] to or from the sweep gas between the point of measurement (P1) of \dot{V}ᵢₙ and a point of measurement (P2) of FCO2ᵢₙ; and/or
calculating a compensated post-oxygenator fraction of CO2 [FCO2_{out,comp}] representing an estimate of a fraction of CO2 at a point of measurement (P3) of \dot{V}ₒᵤₜ, based on FCO2ₒᵤₜ and an estimated addition or removal of water vapour (ΔFH2Oₒᵤₜ) to or from the sweep gas between the point of measurement (P3) of \dot{V}ₒᵤₜ and a point of measurement (P4) of FCO2ₒᵤₜ; and
calculating \dot{V}CO2ₙₑₜ based on at least one of FCO2_{in,comp} and FCO2ₒᵤₜ, comp.

26. The method of claim 24, wherein \dot{V}_{in,comp} is calculated based on FCO2_{in,comp} and the pre-oxygenator fraction of the at least one additional gas, and/or \dot{V}_{out,comp} is calculated based on FCO2ₒᵤₜ, comp and the post-oxygenator fraction of the at least one additional gas.

27. The method of claim 25, wherein \dot{V}_{in,comp} is calculated based on FCO2_{in,comp} and the pre-oxygenator fraction of the at least one additional gas, and/or \dot{V}_{out,comp} is calculated based on FCO2ₒᵤₜ, comp and the post-oxygenator fraction of the at least one additional gas.

28. The method of claim 22, further comprising the steps of:
receiving a target value for the measure of CO2 removal, and
automatically regulating the addition of CO2 to the sweep gas flow so as to reach and/or maintain the target value for the measure of CO2 removal.

29. The method of claim 28, wherein the device is connected to a patient who is also connected to a mechanical ventilator which is configured to mechanically ventilate the patient through the supply of breathing gas to the lungs of the patient, and wherein the target value is selected to evaluate a ventilatory treatment provided by the mechanical ventilator and/or a lung function of the patient, e.g., by selecting a target value corresponding to zero or near zero CO2 removal.

30. The method of claim 21, wherein the step of measuring (S12d) the post-oxygenator sweep gas flow rate (\dot{V}ₒᵤₜ) is performed by measuring a flow rate of a whole effluent flow of sweep gas leaving the oxygenator.

31. The method of claim 29, wherein the oxygenator comprises at least two outlets for sweep gas, the method comprising the steps of:
preventing the sweep gas to pass through all but one outlet of the oxygenator; and
measuring the post-oxygenator sweep gas flow rate (\dot{V}ₒᵤₜ) as the flow rate of the sweep gas flowing through the one outlet.

32. A computer program for controlling carbon dioxide [CO2] removal in a device for extracorporeal blood gas exchange, wherein the device comprises an oxygenator including a membrane acting as a gas-liquid barrier enabling CO2 transfer between a bloodstream and a sweep gas flow through the oxygenator, the computer program comprising computer-readable instructions which, when executed by a control computer, causes the method of claim 21 to be performed.

33. A computer program product comprising a non-transitory memory hardware device storing a computer program for controlling carbon dioxide [CO2] removal in a device for extracorporeal blood gas exchange, wherein the device comprises an oxygenator including a membrane acting as a gas-liquid barrier enabling CO2 transfer between a bloodstream and a sweep gas flow through the oxygenator, the computer program comprising computer-readable instructions which, when executed by a control computer, causes the method of claim 21 to be performed.

34. A system for controlling carbon dioxide [CO2] removal in a device for extracorporeal blood gas exchange, wherein the device comprises an oxygenator including a membrane acting as a gas-liquid barrier enabling CO2 transfer between a bloodstream and a sweep gas flow through the oxygenator, and at least one control computer configured to:
receive a measurement of a pre-oxygenator fraction of CO2 [FCO2ᵢₙ] in the sweep gas flow upstream of the oxygenator;
receive a measurement of a pre-oxygenator sweep gas flow rate (\dot{V}ᵢₙ) of the sweep gas flow upstream of the oxygenator;
receive a measurement of a post-oxygenator fraction of CO2 [FCO2ₒᵤₜ] in the sweep gas flow downstream of the oxygenator;
receive a measurement of a post-oxygenator sweep gas flow rate (\dot{V}ₒᵤₜ) of the sweep gas flow downstream of the oxygenator; and
calculate a net CO2 exchange [\dot{V}CO2ₙₑₜ] over the membrane based on measured FCO2ᵢₙ, \dot{V}ᵢₙ, FCO2ₒᵤₜ and \dot{V}ₒᵤₜ.

35. The system of claim 34, further comprising:
a gas regulator adding CO2 to the sweep gas flow upstream of the oxygenator in order to control a degree of CO2 removal from the bloodstream by the oxygenator,
wherein the control computer is configured to utilize \dot{V}CO2ₙₑₜ as a measure of CO2 removal for regulation of the CO2 addition to the sweep gas flow.

36. The system of claim 35, wherein the control computer is configured to utilize the measure of CO2 removal for regulation of the addition of CO2 to the sweep gas flow by:
causing the measure of CO2 removal to be presented to the operator of the device as decision support in manual adjustment of the addition of CO2 to the sweep gas flow, and/or causing a recommendation for adjustment of the addition of CO2 to the sweep gas flow to be presented to the operator of the device, based on the measure of CO2 removal and a set target for CO2 removal by the oxygenator, and/or
automatically regulating the addition of CO2 to the sweep gas flow based on the measure of CO2 removal.

37. The system of claim 34, wherein the control computer is configured to:
estimate or receive a measurement of a pre-oxygenator fraction of at least one additional gas in the sweep gas flow upstream of the oxygenator, the at least one additional gas being one or more of water vapour (H2O), oxygen [O2], nitrogen gas [N2], and an anaesthetic agent, and/or
estimate or receive a measurement of a post-oxygenator fraction of the at least one additional gas in the sweep gas flow downstream of the oxygenator; and
calculate a compensated pre-oxygenator sweep flow rate [\dot{V}_{in,comp}] based on \dot{V}ᵢₙ, FCO2ᵢₙ and the pre-oxygenator fraction of the at least one additional gas; and/or
calculate a compensated post-oxygenator sweep flow rate [\dot{V}_{out,comp}] based on \dot{V}ₒᵤₜ, FCO2ₒᵤₜ and the post-oxygenator fraction of the at least one additional gas; and
calculate (S2′i) \dot{V}CO2ₙₑₜ based on at least one of \dot{V}_{in,comp} and \dot{V}_{out,comp}.

38. The system of claim 34, wherein the control computer is configured to:
calculate a compensated pre-oxygenator fraction of CO2 [FCO2_{in,comp}] representing an estimate of a fraction of CO2 at a point of measurement (P1) of \dot{V}ᵢₙ, based on FCO2ᵢₙ and an estimated addition or removal of water vapour [ΔFH2Oᵢₙ] to or from the sweep gas between the point of measurement (P1) of \dot{V}ᵢₙ and a point of measurement (P2) of FCO2ᵢₙ, and/or
calculate a compensated post-oxygenator fraction of CO2 [FCO2_{out,comp}] representing an estimate of a fraction of CO2 at a point of measurement (P3) of \dot{V}ₒᵤₜ, based on FCO2ₒᵤₜ and an estimated addition or removal of water vapour [ΔFH2Oₒᵤₜ] to or from the sweep gas between the point of measurement (P3) of \dot{V}ₒᵤₜ and a point of measurement (P4) of FCO2ₒᵤₜ, and
calculate \dot{V}CO2ₙₑₜ based on at least one of FCO2_{in,comp} and FCO2_{out,comp}.

39. The system of claim 37, wherein \dot{V}_{in,comp} is calculated based on FCO2_{in,comp} and the pre-oxygenator fraction of the at least one additional gas, and/or \dot{V}_{out,comp} is calculated based on FCO2ₒᵤₜ and the post-oxygenator fraction of the at least one additional gas.

40. The system of claim 38, wherein \dot{V}_{in,comp} is calculated based on FCO2_{in,comp} and the pre-oxygenator fraction of the at least one additional gas, and/or \dot{V}_{out,comp} is calculated based on FCO2ₒᵤₜ and the post-oxygenator fraction of the at least one additional gas.

41. The system of claim 35, wherein the control computer is configured to:
receive a target value for the measure of CO2 removal, and
regulate the addition of CO2 to the sweep gas flow so as to reach and/or maintain the target value for the measure of CO2 removal.

42. The system of claim 40, wherein the device is connected to a patient who is also connected to a mechanical ventilator which is configured to mechanically ventilate the patient through the supply of breathing gas to the lungs of the patient, and wherein the target value is selected to evaluate a ventilatory treatment provided by the mechanical ventilator and/or a lung function of the patient, e.g., by selecting a target value corresponding to zero or near zero CO2 removal.
